(19) United States
(12) Patent Application Publication
(10) Pub. No.: US 2025/0099219 A1
(43) Pub. Date: Mar. 27, 2025

(54) METHODS OF EMBRYO MULTIPLICATION

(71) Applicant: NBRYO PTY LTD, Yeerongpilly , Queensland (AU)

(72) Inventors: Wilhelmus TE BRAKE, Yeerongpilly, Queensland (AU); Nicholas CAMERON, Yeerongpilly, Queensland (AU); Danielle AZOULAY, Yeerongpilly, Queensland (AU); Margot DAY, Yeerongpilly, Queensland (AU); Michael MORRIS, Yeerongpilly, Queensland (AU)

(21) Appl. No.: 18/834,552

(22) PCT Filed: Jan. 31, 2023
(86) PCT No.: PCT/AU2023/050057

§ 371 Date: Jul. 30, 2024

(30) Foreign Application Priority Data
Jan. 31, 2022 (AU) 2022900164

(51) Int. Cl. A61D 19/04; C12N 5/073; C12N 15/877

(52) U.S. Cl. CPC A61D 19/04; C12N 5/0604; C12N 15/8771; C12N 2509/00

(57) ABSTRACT

The present disclosure relates generally to methods of producing multiple embryos from one or more donor embryos comprising embryonic cells that are developmentally equivalent to embryonic cells from a 16-cell embryo or a pre-compacted morula. More particularly, the method of the disclosure comprises unzipping said donor embryos and expanding one or more aggregates of blastomeres obtained from the donor embryo(s) to produce multiple blastocysts from the donor embryo(s). The present disclosure also relates to the use of such methods in animal breeding.

### CROSS-REFERENCE TO RELATED APPLICATIONS

The present application claims priority from Australian Provisional Patent Application No 2022900164 filed on 31 Jan. 2022, the content of which is incorporated herein by reference in its entirety.

### TECHNICAL FIELD

The present disclosure relates generally to methods of producing multiple embryos from one or more donor embryos comprising embryonic cells that are developmentally equivalent to embryonic cells from a 16-cell embryo or a pre-compacted morula. More particularly, the method of the disclosure comprises unzipping said donor embryos and expanding one or more aggregates of blastomeres obtained from the donor embryo(s) to produce multiple blastocysts from the donor embryo(s). The present disclosure also relates to the use of such methods in animal breeding.

### BACKGROUND

Assisted reproductive technologies (ART) have made tremendous advances, particularly during the past few decades. Artificial insemination (AI) remains the most (cost) effective method for achieving genetic gain in cattle populations and is widely used in the dairy industry. In this regard, the global market remains strong for frozen semen and embryos, with millions of cattle bred by AI, and more than a million embryos transferred annually worldwide. Most of the top sires in the dairy industry that provide semen for AI are derived from embryo transfer (ET), and improvements in methods of controlling the oestrous cycle and ovulation have resulted in more effective programs for AI, superovulation of donor cows, and the management of ET recipients. Notwithstanding these advanced in ART, uptake by producers of reproductive technologies like multiple ovulation and embryo transfer (MOET) remains limited owing to the expense associated with the production of each embryo. Unlike conventional AI, MOET is therefore unlikely to be used by producers as a conventional reproductive method.

More recently, approaches for producing genetically identical monozygotic twins by embryo bisection, as well as from blastomeres separated from cleavage stage embryos, have been reported. Whilst this new addition to the ART “toolbox” is exciting and would enable producers to more effectively capture and select for female (dam) genetics (in addition to sire genetics), the widespread adoption of embryo twinning, like other ET- and IVF-based approaches, at a commercial level is likely to be hampered by the prohibitive cost to the producer, as well as by the challenges associated with scaling of the technology.

Accordingly, there is a need for improved approaches for embryo multiplication to address one or more of these limitations and assist with uptake by industry.

### SUMMARY

The present disclosure is broadly directed to methods of producing multiple embryos from a donor embryo and, in particular, for improving the efficiency with which conceptuses are matured to blastocyst stage post multiplication from a donor. In this regard, the inventors have shown that the efficiency of blastocyst development and maturation is significantly higher for separated bovine blastomeres which are expanded within cell aggregates (e.g., pairs or quads) as compared to blastomeres which are expanded individually, particularly when multiplying donor embryos comprising one or more embryonic cells that are developmentally equivalent to embryonic cells from a 16-cell embryo or a pre-compacted morula. The inventors have also shown that blastocysts matured from embryos produced using the method of the disclosure can give rise to healthy calves when implanted in recipient cows.

In one example, the disclosure provides a method of multiplying one or more donor embryos, said method comprising:
(i) obtaining a donor embryo comprising one or more embryonic cells that are developmentally equivalent to embryonic cells from a 16-cell embryo or a pre-compacted morula;
(ii) separating a plurality of the embryonic cells from the donor embryo;
(iii) expanding the embryonic cells in vitro under conditions suitable to produce a plurality of conceptuses from the donor embryo, wherein at least a portion of the embryonic cells are expanded as one or more cell aggregates, each aggregate comprising 2 or more embryonic cells; and
(iv) culturing the plurality of conceptuses under conditions suitable to produce a plurality of blastocysts.

In one example, separation of the embryonic cells from the or each donor embryo is achieved by disrupting the zona pellucida (ZP), and isolating the embryonic cells from the donor embryo(s). This is referred to herein as “unzipping” or the “unzip method”. In accordance with this example, the ZP may be disrupted and a plurality of embryonic cells isolated from each of the one or more donor embryos. In one example, the ZP is disrupted enzymatically or mechanically. For example, the ZP may be disrupted enzymatically or mechanically, and a micropipette used to aspirate the embryonic cells from the one or more donor embryos, thereby isolating the embryonic cells.

In one example, substantially all of the embryonic cells separated from the donor embryo(s) are expanded as cell aggregates. For example, all of the embryonic cells separated from the donor embryo(s) may be expanded within cell aggregates.

As described herein, the donor embryo comprises one or more embryonic cells that are developmentally equivalent to embryonic cells from a 16-cell embryo or pre-compacted morula. In one example, the donor embryo may comprise 9 or more embryonic cells, wherein at least one of the embryonic cells is developmentally equivalent to an embryonic cell from a 16-cell embryo or pre-compacted morula. For example, the donor embryo may comprise between 9-64 embryonic cells (e.g., such as 9-60 embryonic cells), with the proviso that the embryo has not yet progressed to a blastocyst. For example, the donor embryo may comprise between 16-64 embryonic cells (e.g., such as 16-60 embryonic cells), with the proviso that the embryo has not yet progressed to a blastocyst.

In some examples, the donor embryo may comprise one or more embryonic cells which are developmentally equivalent to embryonic cells from a 32-cell embryo. For example, the donor embryo may be an embryo comprising between 16-32 embryonic cells (e.g., a pre-morula stage embryo). Alternatively, the donor embryo may be a pre-compaction morula comprising about 32-64 embryonic cells (e.g., such as 32-60 embryonic cells), with the proviso that the embryo has not yet progressed to a blastocyst.

The cell aggregates (i.e., aggregate of blastomeres separated from donor embryos) may comprise between about 2-8 embryonic cells prior to expansion. For example, one or more or each cell aggregate may comprise 2-4 embryonic cells prior to expansion. For example, one or more or each cell aggregate may comprise 2 embryonic cells prior to expansion. For example, one or more or each cell aggregate may comprise 4 embryonic cells prior to expansion. For example, one or more or each cell aggregate may comprise 8 embryonic cells prior to expansion.

In one example, the donor embryo comprises 9 or more embryonic cells (e.g., 10, or 11, or 12, or 13, or 14, 15, or 16 or more embryonic cells), wherein at least one of the embryonic cells is developmentally equivalent to an embryonic cell from a 16-cell embryo or pre-compacted morula, and wherein the embryonic cells separated from the donor embryo are expanded within cell aggregates comprised of 2-4 embryonic cells (e.g., 2 or 4 embryonic cells).

In one example, the donor embryo comprises 16 or more embryonic cells (e.g., 16-64 embryonic cells), wherein at least one of the embryonic cells is developmentally equivalent to an embryonic cell from a 16-cell embryo or pre-compacted morula, and wherein the embryonic cells separated from the donor embryo are expanded within cell aggregates comprised of 2-4 embryonic cells (e.g., 2 or 4 embryonic cells). For example, the donor embryo may comprise at least about 16 embryonic cells. For example, the donor embryo may comprise at least about 32 embryonic cells and/or be classified as a 32-cell embryo. For example, the donor embryo may comprise at least about 64 embryonic cells or be classified as a pre-compacted morula which has not yet progressed to a blastocyst.

In one example, at least about 3 conceptuses are produced for each donor embryo at step (iv). In one example, at least about 4 (e.g., such as 5 or 6 or 7 or 8 or 9 or 10 or more) conceptuses are produced for each donor embryo at step (iv). For example, each donor embryo may produce at least 4 conceptuses. For example, each donor embryo may produce at least 5 conceptuses. For example, each donor embryo may produce at least 6 conceptuses. For example, each donor embryo may produce at least 7 conceptuses. For example, each donor embryo may produce at least 8 conceptuses. For example, each donor embryo may produce at least 9 conceptuses. For example, each donor embryo may produce at least 10 conceptuses.

In some examples, a portion of the plurality of embryonic cells separated from the donor embryo are separated as an existing aggregate (e.g., as pairs or quads). Alternatively, or in addition, a portion of the plurality of embryonic cells separated from the donor embryo are aggregated after being separated from the donor embryo. In some examples, the embryonic cells which are aggregated are derived from the same donor embryo. In some examples, the embryonic cells which are aggregated are derived from more than one donor embryo (e.g., such as from more than one embryo from the same animal or from different animals).

In other examples, substantially all of the plurality of embryonic cells separated from the donor embryo are separated within existing aggregates (e.g., as pairs or quads).

In one example, the embryonic cells or conceptuses may be cultured in the presence of one or more factors capable of promoting embryogenesis. For examples, the embryonic cells may be cultured in the presence of one or more factors capable of promoting embryogenesis in order to form and expand the embryos.

In another example, the embryonic cells or embryos comprising same may be cultured in the presence of one or more factors capable of maintaining totipotency and/or inhibiting or preventing embryogenesis. For example, the embryonic cells or embryos comprising same may be cultured in the presence of one or more factors capable of maintaining clearance of maternal mRNAs.

In each of the foregoing examples, the donor embryos may be obtained from a vertebrate animal.

In one example, the vertebrate animal may be a mammalian species.

In one example, the mammalian species may be a livestock species.

In one example, the livestock species may be a ruminant species. For example, the livestock species may be a bovine species. For example, the livestock species may be an ovine species (i.e., sheep). For example, the livestock species may be a caprine species (i.e., goat). For example, the livestock species may be a cervid species (i.e., deer). For example, the livestock species may be a camelid species (e.g., camel or alpaca).

In one example, the livestock species may be a porcine species (i.e., pig).

In one example, the livestock species may be an equine species (i.e., horse).

In some examples, one or more donor embryos obtained at step (i) is/are produced by in vivo fertilisation. In other examples, one or more donor embryos obtained at step (i) is/are produced by in vitro fertilisation (IVF).

In some examples, the donor embryos obtained at step (i) are fresh. In other examples, the donor embryos obtained at step (i) have been cryopreserved. For example, the donor embryos may be thawed. In other examples, the donor embryos obtained at step (i) may have been stored in embryo holding media (e.g., at 4° C.).

In each of the forgoing examples, the method may further comprise selecting one or more of the donor embryos obtained at step (i) on the basis of one or more genetic screening criteria, genetic diagnoses and/or one or more morphological criteria. For example, the selection step may be performed prior to step (i).

In one example, the genetic screening criteria may be determined by screening the one or more donor embryos for the presence or absence of one or more genetic markers (e.g., SNP alleles or haplotype) associated with a trait of interest. In one example, the trait of interest is selected from a phenotypic production trait, drug resistance, susceptibility to pests and/or parasites, and sex (i.e., determining whether the embryo is male or female).

In one example, one or more of the donor embryos may be selected on the basis of a genetic diagnosis for one or more conditions, diseases or predisposition thereto.

In one example, one or more of the donor embryos may be selected on the basis of one or more morphological characteristics which is indicative of embryo health.

In each of the foregoing examples, one or more of the donor embryos may be genetically modified or genetically edited. For example, one or more of the donor embryos may be genetically modified by introducing an exogenous nucleic acid to the genome of the embryonic cells comprised therein. For example, one or more of the donor embryos may be genetically edited by editing the genome of the embryonic cells comprised therein.

In one example, one or more of the donor embryo comprises a unique genetic tag or nucleic acid identifier for traceability of the embryos produced therefrom and/or animals produced from said embryos. For example, the unique genetic tag or nucleic acid identifier may be introduced using genetic modification.

In each of the foregoing examples, the method comprises expanding the plurality of embryos in vitro to form blastocysts. For example, the method may comprise expanding the embryos in vitro to form mature blastocysts which are ready for implantation.

The method may further comprise harvesting the plurality of embryos produced by the method. For example, the method may comprise harvesting the embryos once they mature to the blastocyst stage.

In some examples, one or more of the harvested embryos are stored in an embryo holding medium. For example, the one or more of the harvested embryos may be stored at about 4° C.

In some examples, the one or more of the harvested embryos are cryopreserved. Cryopreserved embryos may be stored at about −180° C. to about −196° C. For example, the cryopreserved embryos may be stored in liquid nitrogen at about −196° C.

In some examples, the method of the disclosure further comprises transferring one or more of the embryos produced by the method to the oviduct(s) or uterus of one of more recipient females.

The present disclosure also provides one or more embryos produced by the method of the disclosure. In one example, the one or more embryos may be provided in embryo storage or transfer media at about 4° C. In another example, the one or more embryos may be cryopreserved.

In one example, the embryo is from a mammalian species (e.g., a non-human mammalian species). In one example, the non-human mammalian species is a livestock species (e.g., a ruminant species). For example, the livestock species may be a bovine species. For example, the livestock species may be an ovine species (i.e., sheep). For example, the livestock species may be a porcine species (i.e., pig). For example, the livestock species may be an equine species (i.e., horse). For example, the livestock species may be a caprine species (i.e., goat). For example, the livestock species may be a cervid species (i.e., deer). For example, the livestock species may be a camelid species (e.g., camel or alpaca).

The present disclosure also provides a method of breeding an animal, comprising:
(i) transferring one or more of the embryos produced by the method of the disclosure to the oviduct(s) or uterus of one of more recipient females to establish a pregnancy;
(ii) producing the animal from the pregnant recipient female by parturition.

In one example, the animal is a vertebrate animal. For example, the vertebrate animal may be a mammal, an amphibian, a reptile, a fish or a bird.

In one particular example, the animal is a mammal (e.g., a non-human mammal). Exemplary non-human mammals which may be produced using the method include livestock species (e.g., cattle, buffalo, pigs, sheep, goats, camelid, deer, horses etc.), companion animals (e.g., dogs, cats etc.), laboratory animals (e.g., rats, mice, hamsters, guinea pigs, rabbits, etc.), non-human primates (macaque and marmoset etc.) and wildlife species (e.g., marsupials, cats, rhino, giant panda, etc.). In one particular example, the method of the disclosure may be used to breed a ruminant livestock species. For example, the method of the disclosure may be used to breed cattle. For example, the method of the disclosure may be used to breed sheep. For example, the method of the disclosure may be used to breed goats. For example, the method of the disclosure may be used to breed cervids. For example, the method of the disclosure may be used to breed camelids. In another example, the method of the disclosure may be used to breed pigs. In yet another example, the method of the disclosure may be used to breed horses.

### BRIEF DESCRIPTION OF THE DRAWING

FIG. 1. Schematic representation of the normal development of preimplantation conceptus development from the zygote stage to the blastocyst stage.

FIG. 2. Schematic representation of classification of blastomeres during a single round of unzipping (Serial N=1) from 8-cell, 16-cell and 32-cell stage conceptuses.

FIG. 3. Multiplication of conceptuses derived from pairs of 1:8 blastomeres obtained by unzipping bovine conceptuses at about the 8-cell stage.

FIG. 4. Multiplication of conceptuses derived from pairs of 1:16 blastomeres obtained by unzipping bovine conceptuses which are at about the 16-cell stage.

FIG. 5. Multiplication of conceptuses derived from quads of 1:32 blastomeres obtained by unzipping bovine conceptuses which are at about the 32-cell stage.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in animal nutrition, feed formulation, microbiology, livestock management).

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

The term “about” is used herein to mean approximately. When the term “about” is used in conjunction with a numerical range, it modifies that range by extending the boundaries above and below the recited numerical values. In general, the term “about” is used herein to modify a numerical value above and below the stated value by 10%, up or down (higher or lower).

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features. Thus, each feature of any particular aspect or embodiment of the present disclosure may be applied mutatis mutandis to any other aspect or embodiment of the present disclosure.

The present disclosure is not to be limited in scope by the specific embodiments described herein, which are intended for the purpose of exemplification only. Functionally equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Specific Definitions

The term “conceptus” typically refers to an embryo from fertilisation (i.e., the zygote that is formed when two haploid gametic cells (e.g., an unfertilized oocyte and a sperm cell) unite to form a diploid totipotent cell (e.g., a fertilized ovum)), until the appearance of the primitive streak, at which time the entity is then referred to as an “embryo”. However, in some instances, the terms “embryo” and “conceptus” are used interchangeably during the time prior to the appearance of the primitive streak. For example, the term “embryo” may also be used to refer to the zygote that is formed at fertilisation, as well as to the embryo that results from the subsequent cell divisions (i.e. embryonic cleavage), including the morula stage (i.e. when the embryo is compacting or has compacted) and blastocyst stage with differentiated trophectoderm and inner cell mass.

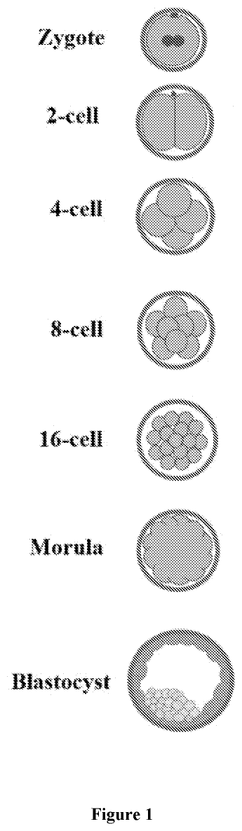

As used herein, the term “morula” refers to a stage of embryonic development. The morula is an early stage embryo that consists of a ball of cells (called blastomeres) contained within a glycoprotein membrane called a zona pellucida. The morula is produced from the single-celled zygote through a series of cleavage events (which is illustrated in FIG. 1 for bovine). A key event prior to morula formation is “compaction”, where the embryo containing about 32-64 cells (depending on species) undergoes changes in cell morphology and cell-cell adhesion that initiates the formation of this solid ball of cells. A “morula” typically comprises around 32-64 cells (depending on species), and resembles a mulberry, hence the name morula (Latin, morus: mulberry). In the context of bovine species, the process of compaction typically takes place after the 16-cell stage and the developing embryo reaches the early morula stage at the 32-cell stage, at which time cell-to-cell adhesion between the embryonic cells (or blastomeres) is progressed.

Through a process involving cellular differentiation and cavitation, the morula gives rise to a blastocyst. As used herein, the term “blastocyst” shall be understood to refer to an embryo which possesses an inner cell mass (ICM), or embryoblast comprising pluripotent embryonic stem cells, and an outer layer of cells, or trophectoderm comprising trophoblasts, which later forms the placenta. The trophectoderm surrounds the inner cell mass and a fluid-filled blastocyst cavity known as the blastocoel. A blastocyst typically comprises between 70-300 embryonic cells (which may vary depending on species and maturity of the embryo). In some examples, a blastocyst may comprise about 64 to about 128 cells. In some examples, a blastocyst may comprise between about 128 to about 256 cells. In some examples, a blastocyst may comprise between 150-256 cells. In some examples, a blastocyst may comprise between about 256-300 cells.

As used herein, the terms “embryonic cell” or “embryonic cells” is intended to encompass all totipotent or pluripotent cells within the developing embryo from the zygote to the blastocyst stage. For example, embryonic cells obtained from within the developing embryo from zygote to morula stage (also referred to as “blastomeres”) are totipotent or pluripotent embryonic cells (depending on the stage of embryogenesis). Likewise, embryonic cells obtained from the inner cell mass of a blastocyst may be pluripotent.

As used herein, the term “totipotent” is used to describe a cell that is capable of giving rise to any cell type. For example, in the context of embryonic cells, a “totipotent” cell is one that can give rise to all of the cell types in an embryo, and ultimately differentiate into any one of the specialised cells required for different tissues in the body (e.g., skin, bone, marrow and muscle etc.). The term “totipotent” is to be distinguished from the term “pluripotent”, the latter referring to cells that differentiate into specific subpopulations of cells within a developing cell mass but which may not give rise to any and all cell types.

A “pluripotent” cell is one that is capable of differentiating to generate primitive ectoderm, which is then able to differentiate during gastrulation into cells of all three germ layers: ectoderm (giving rise to skin and nervous system), endoderm (forming the gastrointestinal and respiratory tracts, endocrine glands, liver, and pancreas) and mesoderm forming bone, cartilage, most of the circulatory system, muscles, connective tissue, and more. A pluripotent cell is also one that is able to self-renew, thereby creating new copies of itself.

As used herein, the term “monozygotic embryos” shall be understood to mean two or more embryos formed or derived from a single zygote.

The term “demi-embryo” as used herein shall be understood to mean a portion of an embryo after it has been separated from the donor embryo. For example, an embryo which is split into two portions of embryonic cells may produce two demi-embryos, each comprising embryonic cells. Likewise, an embryo that is split into three portions, each comprising embryonic cells, may give rise to three demi-embryos. A skilled person will appreciate that embryonic cells may be separated from a donor embryo by any means to give rise to a demi embryo (e.g., “cutting”, “unzipping”, etc.).

As used herein, the term “animal” shall be understood to include all vertebrate animals, such as mammals (i.e., non-human mammals), amphibians, reptile, fish and birds. In one example, the animal is a mammal. Exemplary mammals for which the method of the disclosure may be useful include livestock (e.g., cattle, buffalo, pigs, sheep, goats, camelid, deer, horses etc.), companion animals (e.g., dogs, cats, horses etc.), laboratory animals (e.g., rats, mice, hamsters, guinea pigs, rabbits, etc.), non-human primates (macaque and marmoset etc.) and wildlife species (e.g., marsupials, large cats, rhino, giant panda etc.). In one example, the method of the disclosure may be useful in ruminant livestock species (e.g., cattle, buffalo, sheep, goats, camelid, deer etc). In one particular example, the method of the disclosure may be useful in a bovine species.

### Twinning Methods

The present disclosure is directed generally to methods of multiplying embryos, also referred to herein as “twinning”, and in particular, to methods capable of producing a plurality of embryos from one or more initial donor embryos.

In one example, a method of the disclosure possesses the following general method steps:
(i) obtaining a donor embryo comprising one or more embryonic cells that are developmentally equivalent to embryonic cells from a 16-cell embryo or a pre-compacted morula;
(ii) separating a plurality of the embryonic cells from the donor embryo;
(iii) expanding the embryonic cells in vitro under conditions suitable to produce a plurality of conceptuses from the donor embryo, wherein at least a portion of the embryonic cells are expanded as one or more cell aggregates, each aggregate comprising 2 or more embryonic cells; and
(iv) culturing the plurality of conceptuses under conditions suitable to produce a plurality of blastocysts.

In this regard, the Applicant has discovered unexpectedly that the efficiency with which monozygotic conceptuses can cultured through to the blastocyst stage is significantly increased when embryonic cells separated from the donor embryo are expanded as cell aggregates, as opposed to embryonic cells being expanded individually.

In some examples, all or substantially all of the embryonic cells separated from the donor embryo are expanded as cell aggregates. However, in other examples, a portion of the embryonic cells separated from the donor embryo are expanded as aggregates, whilst other cells may be expanded individually. In this regard, a skilled person may vary the process as desired.

A number of techniques are known in the art for producing a plurality of embryos from a single donor embryo (‘twinning techniques’) and each of these techniques is contemplated herein. Exemplary techniques include “the unzip method” and “the cutting method” or “cut method”, both of which are described in further detail below.

In a preferred example, the ‘twinning technique’ employed in the method of the disclosure is designated “the unzip method”. In accordance with this embodiment, a plurality embryonic cells are separated from the donor embryo by disrupting or “unzipping” the zona pellucida (ZP), and isolating the embryonic cells from within the donor embryo. In accordance with this embodiment, the zona pellucida of the donor embryo is disrupted to release the embryonic cells comprised therein, after which the embryonic cells are isolated (either singularly or as an aggregate or cluster of cells) and expanded in vitro to produce a plurality of embryos according to steps (i)-(iv) above. In this way, each isolated embryonic cell, or each aggregate of embryonic cells (i.e., where two or more cells are isolated together) is expanded to become an embryo (e.g., a ZP-free embryo). As described herein, at least a portion of the embryonic cells which are isolated from each donor embryo are expanded as one or more cell aggregates, wherein each aggregate comprising 2 or more embryonic cells. This assists in improving the efficiency of embryo multiplication.

Disruption of the zona pellucida in the unzip method, also known as “assisted hatching”, may be performed using any suitable method known in the art. In this regard, a variety of techniques are known to be employed to assist embryo hatching in the field of assisted reproduction, including partial mechanical zona dissection, zona drilling and zona thinning, making use of acid tyrodes, proteinases, piezon vibrator manipulators and lasers e.g., as described in Hammadeh et al., (2011) J. Assist. Reprod. Genet., 28 (2): 119-128 It is also contemplated that the zona pellucida may be disrupted by nano-dissection (e.g., using femtosecond laser pulse or atomic force microscopy (AFM) with a nano-scalpel). Any one or more of the above-mentioned techniques may be employed in the unzip method of the disclosure for disrupting the zona pellucida.

Once the zona pellucida is disrupted, embryonic cells (e.g., blastomeres) may be isolated and, if appropriate, transferred to fresh culture media for expansion. A number of methods for isolating individual cells, including embryonic cells, are known in the art and contemplated herein (e.g., as described in Zhu and Murthy (2013) Curr. Opin. Chem. Eng., 2 (1): 3-7;). Techniques for isolating cells include, but are not limited to, fluorescence-activated cell sorting (FACS), magnetic-activated cell sorting (MACS), dielectrophoretic digital sorting, immunomagnetic cell separation, immunosurgery, hydrodynamic traps, laser capture microdissection, mechanical dissection, manual picking, microfluidics, micromanipulation, nanodissection, serial dilution, Raman tweezers and combinations thereof. Any one of these techniques, or a combination thereof, is contemplated for use in the methods of the disclosure to isolate single embryonic cells or aggregates of embryonic cells from the disrupted zona pellucida. In one particular example, microfluidics is employed to isolate individual embryonic cells.

In another example, the ‘twinning technique’ employed in the method of the disclosure is designated “the cutting method” or “cut method”. In accordance with this embodiment, the one or more initial donor embryos at step (ii) are cut (or split) into two or more portions (e.g., three, four or five or more portions), each comprising one or more embryonic cells, and at least one of the portions comprising two or more embryonic cells. In some examples, all of the portions comprise two or more embryonic cells from the donor embryo. The demi-embryos are then expanded in vitro to produce a plurality of embryos (e.g., monozygotic embryos) as set out in steps (i)-(iv) above.

In accordance with embodiments in which the “cutting method” is used, the process of cutting (or splitting) the embryo may be performed using any means known in the art for splitting embryos. For example, the donor embryo may be split or cut by mechanical dissection using microsurgical instruments that rely on pressure, such as a blade (e.g., a scalpel blade or portion thereof) or a fine glass needle. Alternatively, or in addition, the donor embryo may be split or cut using a laser i.e., laser-assisted biopsy. In other examples, the donor embryo may be split or cut using a nano-dissection-based tool (e.g., using femtosecond laser pulse or atomic force microscopy (AFM) with a nano-scalpel). However, it is contemplated that any means known in the art may be employed.

In some examples, steps (i)-(iv) of the embryo multiplication process can be repeated in a serial fashion using the newly produced embryos as the donor embryos. The process can be repeated ‘N’ number of times (each referred to as a cycle), using the embryos produced from the previous cycle as donors embryos for the subsequent cycle. The number of cycles to be performed using the “unzip” method will depend on various factors including the number of embryos to be produced, the number of starting donor embryos, whether or not any embryos are harvested from the method during intervening cycles, and the number of embryonic cells in the donor embryos, the latter determining the upper limit of how many embryos can be produced from any one donor embryo.

Donor embryos for multiplication using the method of the disclosure include embryos comprising one or more embryonic cells that are developmentally equivalent to embryonic cells from a 16-cell embryo or pre-compacted morula. In one example, the donor embryo may comprise 9 or more embryonic cells, wherein at least one of the embryonic cells is developmentally equivalent to an embryonic cell from a 16-cell embryo or pre-compacted morula. For example, the donor embryo may comprise between 9-64 embryonic cells (e.g., 9-60 embryonic cells), with the proviso that the embryo has not yet progressed to a blastocyst. For example, the donor embryo may comprise between 16-64 embryonic cells (e.g., such as 16-60 embryonic cells), with the proviso that the embryo has not yet progressed to blastocyst. In some examples, it may be advantageous to select a donor embryo for multiplication which has a greater number of embryonic cells which are totipotent e.g., a pre-compaction morula comprising about 32-64 embryonic cells (e.g., such as 32-60 embryonic cells), with the proviso that the embryo has not yet progressed to a blastocyst. For example, the donor embryo may comprise one or more embryonic cells which are developmentally equivalent to embryonic cells from a 32-cell embryo. In other examples, the donor embryo may be a pre-morula stage embryo comprising between 16-32 embryonic cells. In each case, a skilled person will appreciate that the number of embryonic cells within the developing embryo at each stage may vary between species.

As described herein, the Applicant has discovered unexpectedly that the efficiency with which monozygotic conceptuses can be cultured through to blastocyst stage following ‘twinning’ is significantly increased when embryonic cells separated from the donor embryo are expanded as cell aggregates (or clusters), as opposed to the embryonic cells being expanded individually. Prior to expansion, it is contemplated that the cell aggregates may comprise any number of embryonic cells from the donor embryos, such as e.g., 2, or 3, or 4, or 5, or 6, or 7, or 8 or more cells per cell aggregate. In some examples, the method comprises expanding aggregates of about 2-4 cells. In other examples, the method comprises expanding aggregates of about 4-6 cells. In other examples, the method comprises expanding aggregates of about 4 cells. However, a skilled person will appreciate that the number of cells in each aggregate may vary according to the outcome and efficiency of multiplication desired.

In one example, the or each donor embryo comprises 9 or more embryonic cells, wherein at least one of the embryonic cells is developmentally equivalent to an embryonic cell from a 16-cell embryo or pre-compacted morula, and at least a portion of the embryonic cells separated therefrom are expanded in cell aggregate comprising 2 or more embryonic cells (e.g., such as about 2-4 cells per aggregate).

In another example, the or each donor embryo comprises 16-64 embryonic cells (e.g., about 16-60 embryonic cells), wherein at least one of the embryonic cells is developmentally equivalent to an embryonic cell from a 16-cell embryo or pre-compacted morula, and at least a portion of the embryonic cells separated therefrom are expanded in cell aggregate comprising 2 or more embryonic cells (e.g., such as about 2-8 cells per aggregate). For example, the method may comprise separating a plurality of embryonic cells from a donor embryo comprising about 16 cells, and expanding the embryonic cells in vitro under conditions suitable to produce a plurality of conceptuses from the donor embryo, wherein at least a portion (or all) of the embryonic cells are expanded as part of one or more cell aggregates comprising about 2 embryonic cells per aggregate prior to expansion. For example, the method may comprise separating a plurality of embryonic cells from a donor embryo comprising about 32 cells, and expanding the embryonic cells in vitro under conditions suitable to produce a plurality of conceptuses from the donor embryo, wherein at least a portion (or all) of the embryonic cells are expanded as part of one or more cell aggregates comprising about 4 embryonic cells per aggregate prior to expansion.

The skilled person will appreciate that the number of conceptuses which are cultured for each donor embryo at step (iv) to produce a plurality of blastocysts will depend on the developmental stage of the donor embryo (i.e., number of cells), as well as the number of cell aggregates separated from the donor. However, it is contemplated that at least about 4 (e.g., such as 5 or 6 or 7 or 8 or 9 or 10 or more) conceptuses will be cultured for each donor embryo at step (iv) to produce a plurality of blastocysts.

In some examples, a portion of the plurality of embryonic cells separated from the donor embryo are separated in existing aggregates (or clusters). Alternatively, or in addition, a portion of the plurality of embryonic cells separated from the donor embryo are aggregated after being separated from the donor embryo. In many instances, the embryonic cells which are aggregated together after being separated from a donor embryo are derived from the same donor embryo. However, in other circumstances, it may be desirable to form aggregates of cells using embryonic cells from multiple donor embryos (e.g., from multiple donor embryos from the same animal or from donor embryos obtained from different animals of the same species). In each of the foregoing examples describing the formation of cell aggregates after separation of cells from the donor embryo(s), the embryonic cells which are aggregated together may be at the same or a similar developmental stage (e.g., cells which are developmentally equivalent to an embryonic cell from a 16-cell embryo may be aggregated together, or cells which are developmentally equivalent to an embryonic cell from a 32-cell embryo may be aggregated together, and so forth). However, in other examples, embryonic cells which are aggregated together may be at differing developmental stages (e.g., cells which are developmentally equivalent to an embryonic cell from an 8-cell embryo may be aggregated with cells which are developmentally equivalent to an embryonic cell from a 16-cell embryo).

In some examples, the embryo multiplication method of the disclosure may be used in conjunction with a serial twinning approach previously developed by the Applicant. For example, prior to culturing the plurality of conceptuses at (iv) to produce the plurality of blastocysts, the method may further comprises the steps of:
(v) isolating one or more of the plurality of conceptuses produced to be used as donor embryos in subsequent multiplications, wherein each donor embryo isolated for subsequent multiplications comprises at least two embryonic cells;
(vi) separating one or more of the embryonic cells from the one or more donor embryos;
(vii) expanding the embryonic cells in vitro under conditions suitable to produce a plurality of conceptuses, each comprising at least two embryonic cells; and
(iv) optionally repeating steps (v)-(vii) ‘N’ times.

Following the desired number of serial multiplications, the plurality of conceptuses may then be cultured under conditions suitable to produce a plurality of blastocysts, in accordance with step (iv) of the method of the disclosure.

As described herein, steps (v)-(vii) of the method may be repeated ‘N’ times in order to produce the desired number of embryos from the original donor embryos. Depending on (1) the number of embryonic cells in the initial donor embryos, (2) the technique(s) used to separate the embryonic cells therefrom and (3) unless stated otherwise, the number of repetitions of steps (v)-(vii) to be performed i.e., ‘N’, may vary. In this regard, and unless stated otherwise, ‘N’ may be ≥1, e.g., 1, or 2, or 3, or 4, or 5, or 6, or 7, or 8, or 9 or 10 or more.

In each of the embodiments of the method described herein, it may be desirable to immobilise the donor embryo(s) in order to cut or split the donor embryos or to enable disruption of the zona pellucida i.e., “unzip” the zona pellucida. Methods of immobilising embryos are known in the art and any one or more of those methods or techniques may be used in the method of the disclosure. Exemplary methods contemplated for use in the method of the disclosure include applying suction to the zona pellucida, making a depression or cul-de-sac in the container, constructing a device that traps the embryo, or making the embryo stick to a surface; e.g., by roughening the surface of the container containing the embryo, using protein-free culture medium or coating the culture container with a material which adheres to the outer membrane of the embryo.

As described herein, embryonic cells or demi-embryos comprising embryonic cells which have been separated from donor embryos using the method of the disclosure are cultured in vitro and expanded to produce a plurality of embryos (e.g., monozygotic embryos).

Methodologies for culturing embryos in vitro at various stages of development are known in the art and contemplated herein. Exemplary methods are described in Examples 1-5 herein. A skilled person will appreciate that the culture conditions are important for growing the developing embryo to a blastocyst stage of development and may be varied/tailored according to the stage of embryonic development, as well as to control rate of embryonic development (e.g., cleavage) to provide sufficient windows of time to perform the multiplications steps of the method of the disclosure. For example, during embryo culture, variables such as temperature and CO₂ levels can be controlled to optimise growth of the developing embryo. For example, the optimum temperature for the development of an embryo is from about 32° C. to about 40° C., preferably from about 35° C. to 39° C., with a temperature of about 37° C. to about 39° C. (e.g., 38.5° C.) being particularly preferred. The optimum CO₂ levels in the culturing environment for the development of an embryo is from about 1% CO₂ to about 10% CO₂, preferably from about 3% CO₂ to about 8% CO₂, and even more preferably about 5% CO₂.

Suitable media for culturing and expanding embryonic cells and embryos are known in the art. For example, culture media that allow embryos to mature to blastocysts at rates comparable with those that occur in vivo are described in Summers and Biggers (2003) Human Reprod Update, 9:557-582. Many of these culture media are based loosely on the concentrations of ions, amino acids, and sugars found in the reproductive tract of the female at the time of egg release, fertilization, and development (Gardner and Lane (1998) Hum Reprod 13:148-160). Typically, culture media containing a phosphate buffer or HEPES organic buffer are used for procedures that involve handling of gametes outside of the incubator, flushing of follicles and micromanipulation. Most culture media utilize a bicarbonate/CO₂ buffer system to keep pH in an suitable range e.g., pH 7.2-7.4. The osmolarity of the culture medium is typically in the range of 275-290 mosmol/kg. Embryos may also be cultured under paraffin oil (or alternative oil which is not toxic to embryos) to prevent evaporation of the medium preserving a constant osmolarity. The oil also minimizes fluctuations of pH and temperature when embryos are taken out of the incubator for microscopic assessment.

Suitable culture medium also typically contains a protein source, such as albumin or synthetic serum that is added at a concentration of about 5 to 20% (w/v or v/v, respectively). Salts may also be added to the medium, such as NaCl, KCl, KH₂PO₄, CaCl₂2H₂O, MgSO₄7H2O, or NaHCO₃. Culture medium also typically contains a carbohydrate source (e.g. glucose) and monocarboxylates (e.g., pyruvate and lactate), since carbohydrates and monocarboxylates are present in the female reproductive tract. Together, they are the main energy source for the developing embryo. Culture media that support the development of zygotes up to 8-cells contain pyruvate and lactate. Some commercial media are glucose free, while others add a very low concentration of glucose to supply the needs of the sperm during conventional insemination. Media that support the development of 8-cell embryos up to the blastocyst stage contain pyruvate and lactate in low concentrations and a higher concentration of glucose. Supplement of the culture medium with amino acids may also be desirable for embryo development. Media that support the development of zygotes up to 8-cells are typically supplemented with non-essential amino acids such as proline, serine, alanine, asparagine, aspartate, glycine, and glutamate. Media that support the development of 8-cell embryos up to the blastocyst stage are also typically supplemented with essential amino acids, such as cysteine, histidine, isoleucine, leucine, lysine, methionine, valine, argentine, glutamine, phenylalanine, threonine, tryptophan. The culture medium may also contain vitamins.

The culture medium may also contain antibiotics. The majority of ART laboratories use culture media containing antibiotics to minimize the risks of microbial growth. The most commonly used antibiotics being Penicillin (β-lactam for Gram-positive bacteria; disturbs cell wall integrity) and Streptomycin (Aminoglycoside for Gram-negative bacteria; disturbs protein synthesis).

Three examples of sequential media for embryo development which may be useful in culturing embryos in the methods of the disclosure are: G1/G2 (Gardner et al, (1998) Hum. Reprod. 13:3434); Universal IVF Medium/MS (Bertheussen et al., (1997); and PI/Blastocyst Medium (Behr et al., (1998) Am. Soc. Rep. Med. 0-262). Media for culturing embryo at different stages of development are commercially available from a range of sources.

Other exemplary culture media for embryo development are described in Examples 1-5 herein and are contemplated for use in the method of the disclosure.

In some examples, the embryonic cells and/or developing embryos are cultured in the presence of one or more factors capable of maintaining totipotency of the embryonic cells and/or inhibiting or preventing embryogenesis. Such factors may be added to culture media in order to prevent or slow down embryogenesis and thereby provide further opportunity to perform additional cycles of steps (i)-(iv) before cellular differentiation starts to occur. Factors which maintain totipotency of embryonic cells and/or which inhibit or prevent embryogenesis are known in the art and contemplated for use herein. For example, factors that maintain totipotency of embryonic cells and/or which inhibit or prevent embryogenesis include anti-miRs and/or ribozymes that block miRNA stability or activity produced by the early embryo. Exemplary anti-miRs may target miRNAs expressed by the embryo which promote clearance of maternal mRNAs (e.g., anti-miRs that target the miR-30 family).

The skilled person will appreciate that the culture conditions may also contribute to maintaining the state of totipotency of the embryonic cells. Accordingly, during culture of the embryonic cells, embryos or demi-embryos, variables such as cell or embryo density, temperature, CO₂ levels and O₂ levels can be controlled to moderate/control the rate of development of the cultured embryos.

As described herein, the method of the disclosure also comprises culturing and expanding the embryos in vitro to form blastocysts, which can then be harvested (e.g., for storage and/or implantation into a recipient female). Accordingly, at some stages of the method, the embryonic cells and/or developing embryos may be cultured in the presence of one or more factors capable of promoting embryogenesis. For example, factors capable of promoting embryogenesis (i.e., embryogenic factors) may be added to culture media used to culture embryos through to blastocyst stage for harvest. Factors which promote embryogenesis are known in the art and contemplated herein.

The skilled person will also appreciate that the culture conditions; e.g., embryo density, temperature and CO₂ levels, can be varied and/or optimised in order to promote embryogenesis.

As described herein, the species of animal from which the donor embryo(s) is/are obtained may be any vertebrate animal, including a species of mammal, a species of amphibian, a species of reptile, a species of fish and a species of bird (e.g., poultry).

In one example, the animal is a mammal (e.g., a non-human mammal). Exemplary non-human mammals for which the method of the disclosure may be useful include livestock species (e.g., cattle, buffalo, pigs, sheep, goats, camelid, deer, horses, etc.), companion animals (e.g., dogs, cats, horses etc.), laboratory animals (e.g., rats, mice, hamsters, guinea pigs, rabbits, etc.), non-human primates (macaque and marmoset, etc.) and wildlife species (e.g., marsupials, cats, rhino, giant panda, etc.).

In one particular example, the method of the disclosure may be used to produce a plurality of embryos (e.g., monozygotic embryos) in a ruminant livestock species. For example, the livestock species may be a bovine species. For example, the livestock species may be an ovine species. For example, the livestock species may be a caprine species. For example, the livestock species may be a cervine species. For example, the livestock species may be a camelid species.

In another example, the method of the disclosure may be used to produce a plurality of embryos (e.g., monozygotic embryos) from pigs. In another example, the method of the disclosure may be used to produce a plurality of embryos (e.g., monozygotic embryos) from goats. In another example, the method of the disclosure may be used to produce a plurality of embryos (e.g., monozygotic embryos) from horses.

Donor embryo used in the first cycle of the method of the disclosure may be prepared in vivo (e.g., by conventionally flushing embryos from a pregnant animal) or by in vitro fertilisation (IVF) methods.

In one example, the donor embryo used in the first cycle of the method is prepared by in vivo methods. For example, an oocyte may be fertilized in vivo (e.g., following copulation or by artificial insemination) and subsequent embryos retrieved from the pregnant female by conventional embryo flushing. In one example, the donor embryos are produced by multiple ovulation embryo transfer (MOET), whereby the donor female is administered hormones prior fertilisation, primarily follicle stimulating hormone (FSH), to stimulate the ovaries of the cycling female animal to induce multiple ovulations.

In another example, the donor embryo used in the first cycle of the method is prepared by in vitro methodologies (i.e., IVF). Methods for producing embryos using IVF are well known in the art. IVF generally involves the production of oocytes from donor animals by follicle aspiration, following by in vitro maturation, fertilisation and culture until the resulting embryos have reached a desired developmental stage. Conveniently, this approach permits the repeated production of embryos from live animals of particular value under controlled conditions. Methods for IVF production of embryos are described in Berlinguer F. “Embryo Production”, In: Animals Production in Livestock, Encyclopedia of Life Support Systems (EOLSS), the full content of which is incorporated herein.

It is also contemplated that the donor embryo used in the first cycle of the method, whether produced by in vivo or in vitro means, may be fresh, stored or thawed (i.e., a thawed cryopreserved embryo). In one example, the donor embryo is fresh. In one example, the donor embryo has been stored in embryo holding media (e.g., at about 4° C.). In another example, the donor embryo is a thawed cryopreserved embryo.

Donor embryos useful in the method of the disclosure may also have undergone genetic modifications. For example, embryonic cells within the donor embryo may be genetically modified prior to performance of the method such that all embryos produced from the donor carry the genetic modification. In one example, the donor embryo is genetically modified by introducing an exogenous nucleic acid to the genome of the embryonic cells comprised therein. The exogenous nucleic acid may be an alternative allele for a gene or loci associated with a trait of interest. Alternatively, the exogenous nucleic acid may be a transgene. In another example, the donor embryo may be genetically modified by editing the genome of the embryonic cells comprised therein (i.e., genome editing). The genome edit may be selected from the group consisting of an insertion, deletion, substitution, inversion or translocation. For example, the genome edit may be an insertion, deletion and/or substitution of a nucleic acid sequence, or one or more nucleotide positions therein, in order to replace an existing allele of a gene or loci associated with a trait of interest with an alternative allele.

Genome editing may also be employed to introduce one or more genetic modifications (e.g., nucleotide substitutions) which, considered alone or in combination, provide a unique genetic profile or fingerprint in the developing embryo. This unique genetic profile or fingerprint can then be used to identify and/or trace embryos produced from the donor embryo (and animals produced therefrom). For example, one or more conservative nucleotide substitutions within safe harbour regions of genome may be made to embryonic cells within the donor embryo in order to generate unique genetic profiles or fingerprints.

Preferably, the genetic modification or editing occurs at the single cell stage such that all subsequent cells in the developing embryo derived from the modified cell (and animal resulting therefrom) comprise the modification. If, however, the genetic modification event occurs after one or more cell divisions, and not all embryonic cells within the donor embryo are modified, then the donor embryo may be a mosaic for the modification/edit event, in that it will have some cells derived from the modified/edited cell and some cells derived from unmodified/unedited cells.

A number of methods for genetically modifying genomes of a cell using targeted nucleases are described in the art. These include but are not limited to (1) clustered regularly interspaced short palindromic repeats (CRISPR)-CRISPR-associated protein 9 (Cas9) or other Cas systems, (2) transcription activator-like effector nucleases (TALENs), (3) zinc-finger nucleases (ZFNs), and (4) homing endonucleases or meganucleases. These are other methods for genetically modifying cells are contemplated for use in the method of the disclosure in order to genetically modify donor embryos.

The methods of the disclosure may further comprise one or more steps to assist with selection of donor embryos to be multiplied using the method. For example, the method may comprise selecting the donor embryo prior to step (i) on the basis of one or more genetic screening criteria, genetic diagnoses and/or one or more morphological criteria.

In one example, genetic screening criteria may be determined by screening for the presence or absence of one or more genetic markers (e.g., SNP alleles or haplotype) associated with a (favourable variant of) phenotypic trait of interest; e.g., a commercially-important production trait as may be the case for a livestock species. Exemplary phenotypic traits of interest include, but are not limited to, production traits (e.g., growth rate, fecundity, feed conversion efficiency, etc.), drug resistance, susceptibility to pests and/or parasites, and sex (i.e., male or female). In this way, donor embryos for multiplication using the method of the disclosure can be obtained from elite animals.

Alternatively, or in addition, the donor embryo may be selected on the basis of a genetic diagnosis for one or more conditions, diseases or predisposition thereto. In this regard, preimplantation genetic diagnosis (PGD) or preimplantation genetic testing (PGT) of embryos has become more common place in the field of IVF. PGD tests have largely focused on two methodologies: fluorescent in situ hybridization (FISH) and polymerase chain reaction (PCR). However, a number of techniques for PGD/PGT are known in the art and one or more of these techniques may be used in the method of the disclosure to select donor embryos. These include, but are not limited to, methods which rely on polymerase PCR, FISH, single strand conformational polymorphism (SSCP), restriction fragment length polymorphism (RFLP), primed in situ labelling (PRINS), comparative genomic hybridisation (CGH), COMET analysis (single cell gel electrophoresis), heteroduplex analysis, Southern analysis, and denaturing gradient gel electrophoresis (DGGE) analysis.

Alternatively, or in addition, the donor embryo may be selected on the basis of one or more morphological characteristics, such as morphological characteristics which are indicative of embryo health.

As described herein, once a desired number of embryos (e.g., monozygotic embryos) are produced using the method of the disclosure, those embryos may be matured to a desired embryonic developmental stage in vitro (e.g., preimplantation blastocyst) and harvested from the culture media. Harvested embryos may then be stored in an appropriate embryo holding or transfer media until they are transferred to recipient females and/or until such a time as the embryo is cryopreserved. Any commercially available embryo holding and transfer media is contemplated for use herein. In accordance with examples in which the embryos are to be placed in short term storage prior to transfer to a recipient female (such as during transport), the harvested embryos may be stored at between about 2° C. to about 8° C. depending on the specifications of the particular holding or transfer media. In some preferred examples, the harvested embryos are stored at about 4° C.

Harvested embryos may also be cryopreserved for storage. The main techniques used in the art for embryo cryopreservation are vitrification and slow programmable freezing (SPF), both of which are contemplated herein. In accordance with this example, the harvested embryos can be transferred to an appropriate cryopreservation media (e.g., containing ethylene glycol freeze media or similar), cryopreserved, and maintained at about −180° C. to about −196° C. until they are thawed for use and/or they are shipped. For example, the cryopreserved embryos may be stored in liquid nitrogen at about −196° C.

In addition to the application of the method of the disclosure in commercial livestock breeding, it is also contemplated that the method of the disclosure may have applications in the area of animal conservation and management. For example, embryos produced from donor embryos obtained from endangered or threatened species (including wildlife and domesticated species) using the method of the disclosure may be deposited with biobanks and/or disseminated for breeding programs. This may assist with breeding programs and management of populations of endangered or threatened species. Accordingly, in some examples, the method may further comprise depositing one or more cryopreserved embryos prepared by the method of the disclosure with a biobank.

In accordance with embodiments in which the harvested embryos are transferred fresh to recipient females, the method of the disclosure may further comprise transferring one or more of the embryos to the oviduct(s) or uterus of one of more recipient females. Whether the embryo is transferred to the uterus or oviduct will depend on the developmental stage of the embryo. Methods for embryo transfer are known in the art. For example, embryos may be manually transferred using a catheter or other means.

### Animal Breeding

The present disclosure also provides a method of breeding an animal, comprising:
(i) transferring one or more of the plurality of embryos produced by the method described herein to the oviduct(s) or uterus of one of more recipient females to establish a pregnancy; and
(ii) producing the animal from the pregnant recipient female by parturition.

As described herein, the animal may be any vertebrate animal, including a species of mammal, a species of amphibian, a species of reptile, a species of fish and a species of bird (e.g., poultry). In one particular example, the animal is a mammal (e.g., a non-human mammal). Exemplary non-human mammals for which the method of the disclosure may be useful include livestock species (e.g., cattle, buffalo, pigs, sheep, goats, camelid, deer, horses etc.), companion animals (e.g., dogs, cats etc.), laboratory animals (e.g., rats, mice, hamsters, guinea pigs, rabbits, etc.), non-human primates (macaque and marmoset etc.) and wildlife species (e.g., marsupials, cats, rhino, giant panda etc.). In one particular example, the method of the disclosure may be used to breed cattle. In another example, the method of the disclosure may be used to breed sheep. In another example, the method of the disclosure may be used to breed pigs. In another example, the method of the disclosure may be used to breed goats. In another example, the method of the disclosure may be used to breed horses. In another example, the method of the disclosure may be used to breed camelids (e.g., alpacas).

### EXAMPLES

### Example 1: Multiplication of Bovine Conceptuses

### 1.1 General Methods and Materials

### 1.1.1 Conceptus Culture Media Preparation

### 1.1.1.1 Chemicals and Stock Solutions

Stock solutions for unzipping medium were prepared according to Table 1. Unless described otherwise, medium reagents used in this study were obtained from Sigma. Stock solutions were prepared using Milli-Q® water. The vapor pressure osmometer (Wescor) was used to adjust the osmolality of NaCl, KCl and NaHCO₃ to 2000 mOsm, 200 mOsm and 2000 mOsm respectively, using Milli-Q® water.

TABLE 1
Stock used to medium for unzipping.

| | Final | Amount/volume | |
|---|---|---|---|
| Compound | concentration | of H₂O | Storage |
| NaCl | 1M | 2920 mg/50 mL | Stored at 4° C. |
| KCl | 0.1M | 370 mg/50 mL | Stored at 4° C. |
| Na₂EDTA | 0.001M | 19 mg/50 mL | Stored at 4° C. |
| HEPES | 0.154M | 1835 mg/50 mL | Stored at 4° C. |
| Myo-inostiol | 0.277M | 998 mg/20 mL | Aliquoted and |
| | | | stored at −20° C. |
| Na₃citrate•2H₂0 | 0.05M | 294 mg/20 mL | Aliquoted and |
| | | | stored at −20° C. |
| KH₂PO₄ | 0.1M | 136 mg/10 mL | Stored at 4° C. |
| MgSO₄ | 0.1M | 246 mg/10 mL | Stored at 4° C. |
| NaOH | 2M | 800 mg/10 mL | Stored at 4° C. |
| HCl | 1M | 1 mL/10 mL | Stored at 4° C. |
| NaHCO₃ | 1M | 420 mg/5 ml | Freshly made for |
| | | | one week use |
| Na Pyruvate | 0.1M | 55 mg/5 mL | Freshly made for |
| | | | one week use |
| Glucose | 0.1M | 90 mg/5 ml | Freshly made for |
| | | | one week use |
| Gentamicin | 5 mg/mL | 25 mg/5 mL | Freshly made for |
| | | | one week use |

### 1.1.1.2 Preparation of Medium for Unzipping

NbryoIVC-2 Ca²⁺-free medium was used for conceptus unzipping procedures. Medium was prepared by adding stock solutions (from Table 1) in the order listed in Table 2. The pH of the medium was adjusted to 7.4 by adding 2 M NaOH. Medium was tested for osmolality using an osmometer (Wescor). Osmolality was adjusted to 270 mOsm by the addition of Milli-Q® water. Lastly, fatty acid free bovine serum albumin (FAF-BSA) was added to medium at a concentration of 4 mg/mL and medium was filter-sterilized with a 0.22 μm syringe filter (Millipore). Medium was stored at 4° C. for a maximum of two weeks.

TABLE 2
Composition of NbryoIVC-2 Ca²⁺-free medium
made from stock solutions in Table 1.

| Component | Final concentration | |
|---|---|---|
| Lactic Acid (60%) | 4.92 | mM |
| NaCl | 100 | mM |
| KCl | 6.64 | mM |
| KH₂PO₄ | 1.10 | mM |
| MgCl₂•6H₂O | 0.45 | mM |
| NaHCO₃ | 23.27 | mM |
| Na₂EDTA | 0.1 | mM |
| Na Pyruvate | 0.4 | mM |
| Gentamicin | 0.05 | mg/mL |
| Myo-inositol | 2.56 | mM |
| Na₃-citrate | 0.46 | mM |
| H₂O | 20 | mL |

### 1.1.1.3 Pronase Preparation

Pronase is a proteolytic enzyme used for the removal of the zona pellucida (ZP) during unzipping procedures. Pronase was prepared at a final concentration of 0.3 mg/mL in HEPES buffered-SOF (synthetic oviductal fluid) medium. It was then filter-sterilized through a 0.22 μm syringe filter, aliquoted, and stored at −20° C.

### 1.1.2. In Vitro Development of Bovine Conceptuses

### 1.1.2.1 Production of Bovine Zygotes

Bovine zygotes were produced by IVM and IVF using commercial protocols of Art Lab Solutions. Bovine oocytes were matured for 24 h in vitro (IVM) from ovaries collected from Nindooinbah Cattle Farm following standard procedures. Matured oocytes were then fertilized for 24 h in vitro with thawed semen from single or multiple bulls of proven fertility from Nindooinbah Cattle Farm. Following IVF, the presumptive zygotes were moved to VitroCleave (Art Lab Solutions) in vitro culture (IVC) medium. Unless stated otherwise, zygotes were cultured in VitroCleave (Art Lab Solutions) IVC medium at 38.5° C. under 5% O₂ and 5% CO₂. Zygotes were cultured to about the 8-, 16- or 32-cell stage 96-100 h after IVF.

### 1.1.3. Preparation of Plates and Dishes for Unzipping

1.1.3.1 Pre-Coating Plates with 0.1% PVA

To avoid adherence of ZP-free conceptuses to the plate, wells of 96-well round bottom plates (Corning) were coated with PVA by adding 50 μL sterile 0.1% PVA (w/v) to each well and allowed to incubate overnight at 38.5° C. Each well was then washed three times with sterile water to remove unbound PVA. The plates were then dried, sealed and stored at 4° C. until use.

### 1.1.3.2 Unzipping Dishes

Prior to the unzipping procedure, a 55 mm Petri dish (Corning) containing 20 μL separate droplets of pronase, NbryoIVC-2 Ca²⁺-free medium, and VitroBlast (Art Lab Solutions) media overlaid with mineral oil (Coopers Scientific), were equilibrated at 38.5° C. under 5% 02, 5% CO₂ for at least 60 min.

### 1.1.3.3 Post-Unzipping Culture System

The 96-well plates pre-coated with 0.1% PVA were utilized for the culture of blastomeres after unzipping. Each well contained 20 μL VitroBlast medium (Art Lab Solutions) overlaid with mineral oil, to avoid medium evaporation. Plates with medium were equilibrated for at least 60 min at 38.5° C. under 5% 02, 5% CO₂ prior to transferring blastomeres into each well.

### 1.1.4. Unzipping Procedure

All bovine conceptuses underwent a single unzipping procedure (serial N=1) which was performed under a dissecting microscope with a plate heated to 37° C. Conceptuses at about the 8-cell, 16-cell or 32-cell stage were treated with pronase, to remove the surrounding ZP, for 2 min at 38.5° C. in a humidified incubator in an atmosphere of 5% 02 and 5% CO₂. Once the ZP dissolved, conceptuses were washed through three 20 μL drops of VitroBlast medium (Art Lab Solution) to rinse off any remaining pronase and incubated for 10 min at 38.5° C. under 5% 02 and 5% CO₂. ZP-free conceptuses were then transferred to NbryoIVC-2 Ca²⁺-free medium for 3 min at 38.5° C. under 5% 02 and 5% CO₂ to decrease cell-cell contact. Then, in the NbryoIVC-2 Ca²⁺-free medium blastomeres in each conceptus were separated by aspiration using a micropipette (˜120 μm diameter). Blastomeres were transferred individually, in pairs or in quads to PVA pre-coated wells containing VitroBlast medium (Art Lab Solutions) and cultured under 5% 02, 5% CO₂, at 38.5° C. In addition, individual blastomeres were also placed together to form aggregate pairs or quads within the well. On some occasions blastomere aggregates were formed from different donor conceptuses. Blastomeres were observed every 12-24 h until the blastocyst equivalent stage and scored for their developmental status (cleaved, compacted, cavitated and blastocyst equivalent). Conceptuses were classified as a blastocyst equivalent when the cavity was greater than half the volume of the conceptus and there was a cohesive cluster of ICM cells.

### 1.1.5. Nomenclature

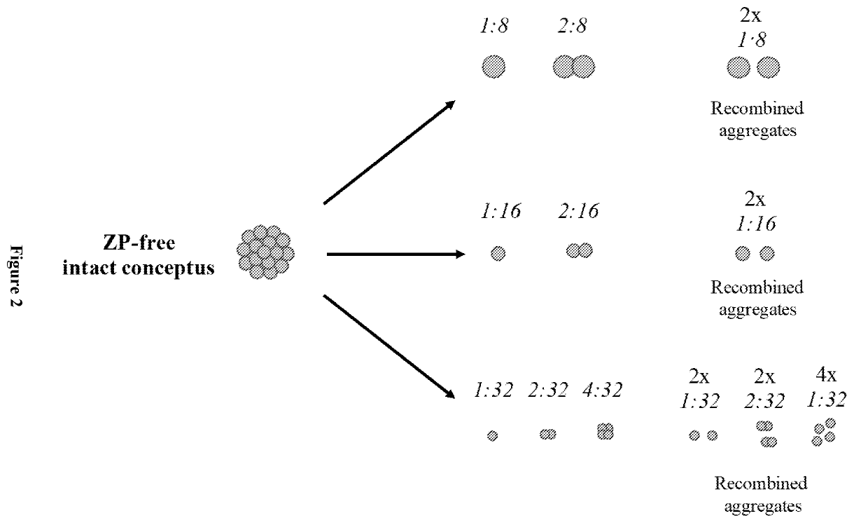

Individual blastomeres isolated from about the 8-, 16-, 32-cell stage conceptuses are referred as 1:8, 1:16, 1:32, respectively. The number before the colon denotes the number of blastomeres and the number after the colon denotes the equivalent conceptus stage the blastomere is at when obtained by unzipping. As a result, pairs and quads of blastomeres are denoted as 2 and 4, respectively, before the colon (e.g., 2:8, 2:16, 2:32 and 4:32). In addition, individual blastomeres from about the 8-, 16- and 32-cell stage conceptuses that were placed together to form pairs are referred as 2× 1:8, 2× 1:16, 2× 1:32, respectively. Formation of quads of blastomeres obtained from about the 32-cell stage conceptuses via recombination of four individual or two pairs of blastomeres are referred as 4× 1:32 or 2× 2:32. FIG. 2 portrays this nomenclature system with regards to the unzipping of conceptuses via serial N=1 unzipping. As shown in FIG. 2, during the unzipping procedure, the zona pellucida (ZP)-free intact conceptus is separated to individual, pairs or quads of blastomeres within the conceptus. Cleavage of cells in the conceptus is not synchronous, and heterogenous stages of blastomere development exist in these conceptuses, which can comprise of 1:8, 1:16 and 1:32 blastomeres. Pairs and quads of unzipped blastomeres are denoted as 2 and 4, respectively, before the colon (e.g., 2:8, 2:16, 2:32 and 4:32). In addition, formation of aggregates can be attained by recombining individual blastomeres (e.g., 2× 1:8, 2× 1:16, 2× 1:32 and 4× 1:32) or by combining pairs (2× 2:32).

### 1.2 Results

Bovine conceptuses at approximately the 8-cell, 16-cell and 32-cell stage were subjected to a single unzipping procedure. Since cleavage of cells in the conceptus is not synchronous, heterogenous stages of blastomere development exist in these conceptuses, comprising of 1:8, 1:16 and 1:32 blastomeres. As a result, each type of blastomere was analyzed separately.
1.2.1 Formation of Blastocyst Equivalents from Singles and Pairs of 1:8 Blastomeres

From 6 replicate experiments, 186 1:8 blastomeres were obtained by unzipping, of which 37 (19.2%) formed blastocyst equivalents (Table 3). The unzipped conceptuses were also separated into pairs of blastomeres (2:8). From 6 replicates, 32 2:8 were obtained, of which 9 (28.1.5%) formed blastocyst equivalents. Additionally, individual 1:8 blastomeres were cultured together to form an aggregate pair (2× 1:8). From 5 replicates, 81 recombined pairs were obtained, of which 21 (25.9%) formed blastocyst equivalents. Individual 1:8 blastomeres from different conceptuses were also recombined to form aggregate pair. From 3 replicates, 6 2× 1:8 were obtained, of which 1 (16.7%) blastocyst equivalents formed.

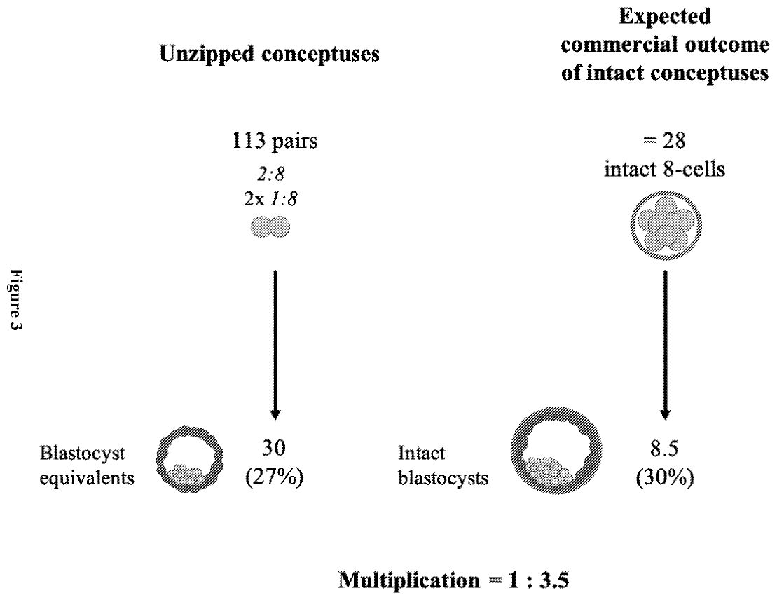

Intact conceptuses can be cultured to the blastocyst stage with a typical efficiency of ˜30%, as reported in standard commercial IVF procedures. The left hand side of FIG. 3 demonstrates the formation of blastocyst equivalents from unzipped pairs of blastomeres (2:8 and 2× 1:8). There were 113 pairs (32 2:8+81 2× 1:8) blastomeres obtained from the donor conceptuses, of which 30 formed blastocyst equivalents (27%). The right hand side demonstrates the expected commercial outcome of intact conceptuses in relation to the 113 pairs of unzipped conceptuses; 113 unzipped pairs is equivalent to 28 intact 8-cell conceptuses. From 28 intact conceptuses, 8.5 intact blastocysts are expected to form (30%; as reported in commercial procedures). As a result, the unzipping procedure enables a multiplication of 3.5 times the rate of blastocyst development when compared to standard commercial procedures.

TABLE 3
Development of blastocyst equivalents from singles
and pairs of 1:8 blastomeres obtained by unzipping
bovine conceptuses at the ~8-cell stage.

| Blastomere Type | 1:8 | 2:8 | 2x 1:8 |
|---|---|---|---|
| Total # obtained | 186 | 32 | 81 |
| Total # blastocyst | 37 | 9 | 21 |
| equivalents (%) | (19.2%) | (28.1%) | (25.9%) |
| # experiment | 7 | 6 | 5 |
| replicates | | | |

1.2.2 Formation of Blastocyst Equivalents from Singles and Pairs of 1:16 Blastomeres

From 6 experiment replicates, 352 1:16 blastomeres were obtained by unzipping, of which 39 (11.1%) formed blastocyst equivalents (Table 4). The unzipped conceptuses were also separated into pairs of blastomeres (2:16). From 7 replicates, 538 2:16 were obtained, of which 277 (51.5%) formed blastocyst equivalents. Additionally, individual 1:16 blastomeres were cultured together to form a pair (2× 1:16). From 5 replicates, 553 recombined pairs were obtained, of which 238 (43.0%) formed blastocyst equivalents. Individual 1:16 blastomeres from different conceptuses were also recombined to form aggregate pairs. From 3 replicates, 15 2× 1:16 were obtained, of which 4 (26.7%) blastocyst equivalents formed. As can be seen from the data in Table 4, the expansion of blastomere pairs from 16-cell conceptuses (2× 1:16) yielded significantly more blastocyst equivalents relative to 16-cell blastomeres expanded individually.

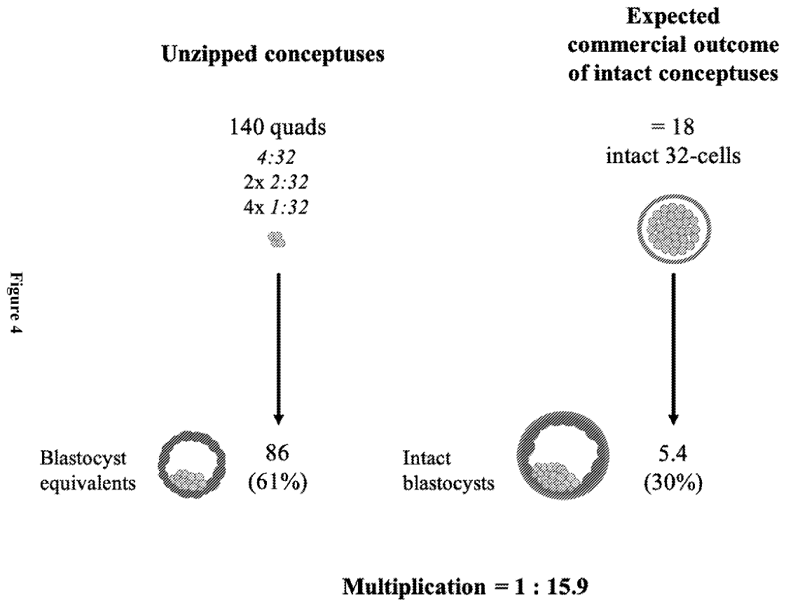

The left hand side of FIG. 4 demonstrates the formation of blastocyst equivalents from unzipped pairs of blastomeres (2:16 and 2× 1:16). There were 1091 pairs (538 2:16+553 2× 1:16) blastomeres obtained, of which 515 formed blastocyst equivalents (47%). The right hand side demonstrates the expected commercial outcome of intact conceptuses in relation to the 1091 pairs of unzipped conceptuses; 1091 unzipped pairs is equivalent to 136 intact 16-cell conceptuses. From 136 intact conceptuses, 40.9 intact blastocysts are expected to form (30%; as reported in commercial procedures). As a result, the unzipping procedure enables a multiplication of 12.6 times the rate of blastocyst development when compared to standard commercial procedures.

TABLE 4
Development of blastocyst equivalents from singles
and pairs of 1:16 blastomeres obtained by unzipping
bovine conceptuses at the ~16-cell stage.

| Blastomere Type | 1:16 | 2.16 | 2x1:16 |
|---|---|---|---|
| Total # obtained | 351 | 538 | 553 |
| Total # blastocyst | 39 | 277 | 238 |
| equivalents (%) | (11.1%) | (51.5%) | (43.0%) |
| # experiment | 6 | 7 | 5 |
| replicates | | | |

1.2.3 Formation of Blastocyst Equivalents from Singles, Pairs and Quads of 1:32 Blastomeres

From 3 experiment replicates, 27 1:32 blastomeres were obtained by unzipping, of which none formed blastocyst equivalents (Table 5). The unzipped conceptuses were also separated into pairs of blastomeres (2:32). From 7 replicates, 71 2:32 were obtained, of which 28 (39.4%) formed blastocyst equivalents. Additionally, individual 1:32 blastomeres were cultured together to form a pair (2× 1:32). From 5 replicates, 46 recombined pairs were obtained, of which 6 (13.0%) formed blastocyst equivalents. Individual 1:32 blastomeres from different conceptuses were also recombined to form aggregate pair. From one replicate, 1 2× 1:32 was obtained, of which 1 (100.0%) blastocyst equivalent formed.

Groups of four blastomeres (quads) were also assessed. Five replicates were performed on each of 4:32, 2× 2:32 and 4× 1:32 blastomere groups. In the 4:32 group, 34 blastomeres were obtained, of which 24 (70.6%) blastocyst equivalents formed. In the 2× 2:32 group, 46 blastomeres were obtained, of which 25 (54.3%) blastocyst equivalents formed. In the 4× 1:32 group, 60 blastomeres were obtained, of which 37 (61.7%) blastocyst equivalents formed. Individual 1:32 blastomeres from different conceptuses were also recombined to form aggregate quad. From one replicate, one 4-cell aggregate comprised of 1:32+3:32 was obtained, of which 1 (100.0%) blastocyst equivalent formed.

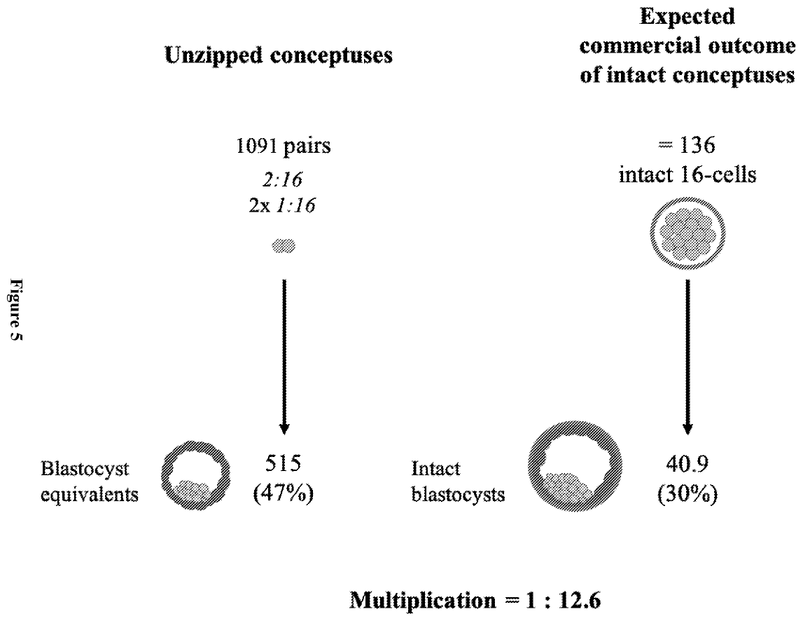

The left hand side of FIG. 5 demonstrates the formation of blastocyst equivalents from unzipped quads of blastomeres (4:32, 2× 2:32, 4× 1:32). There were 140 quads (34 4:32+46 2× 2:32+60 1× 4:32) blastomeres obtained, of which 86 formed blastocyst equivalents (61%). The right hand side demonstrates the expected commercial outcome of intact conceptuses in relation to the 140 quads of unzipped conceptuses; 140 unzipped pairs is equivalent to 19 intact 32-cell conceptuses. From 18 intact conceptuses, 5.4 intact blastocysts are expected to form (30%; as reported in commercial procedures). As a result, the unzipping procedure enables a multiplication of 15.9 times the rate of blastocyst development when compared to standard commercial procedures.

TABLE 5
Development of blastocyst equivalents from singles, pairs and quads of 1:32
blastomeres obtained by unzipping bovine conceptuses at the ~32-cell stage.

| Blastomere Type | 1:32 | 2:32 | 2x 1:32 | 4:32 | 2x 2:32 | 4x 1:32 |
|---|---|---|---|---|---|---|
| Total # obtained | 27 | 71 | 46 | 34 | 46 | 60 |
| Total # blastocyst | 0 | 28 | 6 | 24 | 25 | 37 |
| equivalents (%) | (0.0%) | (39.4%) | (13.0%) | (70.6%) | (54.3%) | (61.7%) |
| # experiment | 3 | 7 | 5 | 5 | 5 | 5 |
| replicates | | | | | | |

### Example 2: Transfer of Blastocyst Equivalents Derived from 2:16 and 4:32 Aggregates

### 1.1 General Methods and Materials

### 1.1.1 Synchronization of Cow Recipients

Estrous cycle was synchronized for transfer of conceptuses on Day 7 or Day 8 using standard protocol of hormonal treatment. Recipient were examined by transrectal palpation for the presence of corpus luteum and conceptuses were transferred to the uterine horn ipsilateral to the ovary containing the corpus luteum.

### 1.1.2 Transfer of Blastocyst Equivalents to Recipient Cows

Blastocyst equivalents were derived from aggregates of either 2:16 or 4:32 as described in Example 1 (multiplication of bovine conceptuses). Day 7 and Day 8 blastocyst equivalents were scored for the presence of inner cell mass (ICM). Blastocyst equivalents with ICM were placed in a 0.25 mL straw either individually or with a blastocyst equivalent with no visible ICM in holding medium (Transport VitroBlast Art Lab Solutions).

### 1.1.3 Pregnancy Diagnosis

Pregnancies were determined either by blood test (Idexx) or by transrectal ultrasound at 2-3 weeks and 1-3 months post-transfer.

### 1.2 Results

1.2.1 Pregnancy and Calving Rate of Blastocyst Equivalent Derived from 2:16 and 4:32 Aggregates

Blastocyst equivalents derived from either 2:16 or 4:32 aggregates were transferred to total of 140 recipients cows in two replicate experiments. The pregnancy rates at 2-3 weeks post-transfer, and 1-3 months post-transfer are shown in Table 2.1. A total of 12 calves were born out of 60 recipients (Table 2.1).

TABLE 2.1
Pregnancy and calving rate of blastocyst equivalent
derived from 2:16 and 4:32 aggregates.

| | | | | | Pregnancy | Pregnancy | |
|---|---|---|---|---|---|---|---|
| | | | | | at 2-3 | at 1-3 | |
| | | | | | weeks | months | |
| Blastocyst | | Blastocyst | | | post- | post- | Calving |
| equivalent | Recipient | equivalent | Replicate | No. of | transfer | transfer | rate |
| Day | Day | type | no. | recipient | (%) | (%) | (%) |
| Day 7 | Day 7 | 2:16 | 1 | 14 | 4/14 | 4/14 | 4/14 |
| | | | | | (29%) | (29%) | (29%) |
| | | | 2 | 13 | 4/13 | 3/13 | TBD |
| | | | | | (31%) | (23%) | |
| | | 4:32 | 1 | 2 | 0/2 | 0/2 | 0/2 |
| | | | | | (0%) | (0%) | (0%) |
| | | | 2 | 9 | 4/9 | 2/9 | TBD |
| | | | | | (44%) | (22%) | |
| Day 8 | Day 7 | 2:16 | 1 | 14 | 0/14 | 0/14 | 0/14 |
| | | | | | (0%) | (0%) | (0%) |
| | | | 2 | 16 | 3/16 | 2/16 | TBD |
| | | | | | (19%) | (13%) | |
| | | 4:32 | 1 | 9 | 4/9 | 4/9 | 4/9 |
| | | | | | (44%) | (44%) | (44%) |
| | | | 2 | 9 | 3/9 | 1/9 | TBD |
| | | | | | (33%) | (11%) | |
| Day 8 | Day 8 | 2:16 | 1 | 17 | 3/17 | 3/17 | 3/17 |
| | | | | | (18%) | (18%) | (18%) |
| | | | 2 | 19 | 3/19 | 3/19 | TBD |
| | | | | | (16%) | (16%) | |
| | | 4:32 | 1 | 4 | 1/4 | 1/4 | 1/4 |
| | | | | | (25%) | (25%) | (25%) |
| | | | 2 | 14 | 3/14 | 2/14 | TBD |
| | | | | | (21%) | (14%) | |

TBD = to be determined

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of multiplying a donor embryo, said method comprising:
(i) obtaining a donor embryo comprising one or more embryonic cells that are developmentally equivalent to embryonic cells from a 16-cell embryo or a pre-compacted morula;
(ii) separating a plurality of the embryonic cells from the donor embryo;
(iii) expanding the embryonic cells in vitro under conditions suitable to produce a plurality of conceptuses from the donor embryo, wherein at least a portion of the embryonic cells are expanded as one or more cell aggregates, each aggregate comprising 2 or more embryonic cells; and
(iv) culturing the plurality of conceptuses under conditions suitable to produce a plurality of blastocysts.

2. The method of claim 1, wherein separating the one or more of the embryonic cells from the one or more donor embryos is achieved by disrupting the zona pellucida (ZP), and isolating the embryonic cells from the one or more donor embryos.

3. The method of claim 2, wherein the ZP is disrupted enzymatically or mechanically, and a micropipette is used to aspirate the one or more of the embryonic cells and/or aggregates thereof from the one or more donor embryos thereby isolating the embryonic cells.

4. The method of any one of claims 1 to 3, wherein all or substantially all of the embryonic cells separated from the donor embryo are expanded as cell aggregates.

5. The method of any one of claims 1 to 4, wherein the donor embryo comprises between about 9-64 cells and wherein the embryo has not yet progressed to a blastocyst.

6. The method of any one of claims 1 to 5, wherein the donor embryo comprises between about 16-64 cells and wherein the embryo has not yet progressed to a blastocyst.

7. The method of any one of claims 1 to 6, wherein the donor embryo comprises between about 32-64 cells and wherein the embryo has not yet progressed to a blastocyst.

8. The method of any one of claims 1 to 4 wherein the donor embryo comprises one or more embryonic cells which are developmentally equivalent to embryonic cells from a 32-cell embryo.

9. The method of any one of claims 1 to 8, wherein each cell aggregate comprises 2-8 embryonic cells prior to expansion.

10. The method of claim 9, wherein each cell aggregate comprises 2-4 embryonic cells prior to expansion.

11. The method of claim 10, wherein each cell aggregate comprises 4 embryonic cells prior to expansion.

12. The method of any one of claims 1 to 11, wherein at least about 4 conceptuses are cultured for each donor embryo at step (iv) to produce a plurality of blastocysts.

13. The method of claim 12, wherein at least about 6-8 conceptuses are cultured for each donor embryo at step (iv) to produce a plurality of blastocysts.

14. The method of any one of claims 1 to 13, wherein:
(i) at least a portion of the plurality of embryonic cells separated from the donor embryo are separated as an existing aggregate; or
(ii) at least a portion of the plurality of embryonic cells separated from the donor embryo are aggregated after being separated from the donor embryo.

15. The method of claim 14, wherein the embryonic cells which are aggregated are derived from the same donor embryo or from more than one donor embryo.

16. The method of claim 15, wherein the embryonic cells which are aggregated are derived from donor embryos obtained from different animals.

17. The method of any one of claims 1 to 16, wherein:
(i) the embryonic cells are cultured in the presence of one or more factors capable of promoting embryogenesis; and/or
(ii) the embryonic cells are cultured in the presence of one or more factors capable of maintaining totipotency.

18. The method of any one of claims 1 to 17, wherein the donor embryos are from a mammalian species.

19. The method of claim 18, wherein the mammalian species is:
(i) a livestock species;
(ii) a ruminant species; and/or
(iii) a bovine, ovine or caprine species.

20. The method of any one of claims 1 to 19, wherein the one or more donor embryos at step (i) is/are produced by in vivo fertilisation (IVF).

21. The method of any one of claims 1 to 20, wherein:
one or more donor embryos at step (i) are fresh;
one or more donor embryos at step (i) have been stored in embryo holding media; and/or
one or more donor embryos at step (i) have been cryopreserved.

22. The method of any one of claims 1 to 21, further comprising selecting the one or more donor embryos prior to step (i) on the basis of one or more genetic screening criteria, genetic diagnoses and/or one or more morphological criteria.

23. The method of any one of claims 1 to 22, wherein the one or more donor embryos have been genetically modified.

24. The method of claim 23, wherein the one or more donor embryos comprise a unique genetic tag or identifier for traceability of the embryos produced therefrom and/or animals produced from said embryos.

25. The method of any one of claims 1 to 24, wherein the plurality of embryos produced are expanded in vitro to form blastocysts.

26. The method of any one of claims 1 to 25, further comprising harvesting the embryos produced by the method.

27. The method of claim 26, wherein one or more of the harvested embryos are stored in an embryo holding media and/or stored at 4° C.

28. The method of claim 27, wherein one or more of the harvested embryos are cryopreserved.

29. The method of any one of claims 1 to 28, further comprising transferring one or more of the embryos produced by the method to the uterus or oviduct(s) of one of more recipient females.

30. The method of any one of claims 1 to 29, wherein prior to the step of culturing the plurality of conceptuses to produce the plurality of blastocysts at (iv), the method further comprises:
(v) isolating one or more of the plurality of conceptuses produced at (iii) to be used as donor embryos in subsequent multiplications, wherein each donor embryo isolated for subsequent multiplications comprises at least two embryonic cells;
(vi) separating one or more of the embryonic cells from the one or more donor embryos;
(vii) expanding the embryonic cells in vitro under conditions suitable to produce a plurality of conceptuses, each comprising at least two embryonic cells; and
(viii) optionally repeating steps (i)-(iii) ‘N’ times, optionally wherein ‘N’ is between 1-10.

31. One or more embryos produced by the method of any one of one of claims 1 to 30.

32. A method of breeding an animal, comprising:
(i) transferring one or more of the embryos produced by the method of any one of claims 1 to 30 to the uterus or oviduct(s) of one of more recipient females to establish a pregnancy; and
(ii) producing the animal from the pregnant recipient female by parturition.

33. The method of claim 32, wherein:
(i) the animal is a mammalian species;
(ii) the mammalian species is a livestock species;
(iii) the livestock species is a ruminant species; and/or
(iv) the livestock species is a bovine, ovine or caprine species.
